(19) United States
(12) Patent Application Publication
(10) Pub. No.: US 2025/0283140 A1
(43) Pub. Date: Sep. 11, 2025

(54) Deep Eutectic Solvent Stabilized Enzyme-Based Electrochemical Assay Probes for Use in the Field

(71) Applicant: The Government of the United States of America, as represented by the Secretary of the Navy, Arlington, VA (US)

(72) Inventors: Scott A. Trammell, Springfield, VA (US); Jeffrey S. Erickson, Arlington, VA (US); David A. Stenger, Annapolis, MD (US); Jeremiah Smith, Vienna, VA (US)

(21) Appl. No.: 18/599,563

(22) Filed: Mar. 8, 2024

(51) Int. Cl. C12Q 1/00

(52) U.S. Cl. CPC C12Q 1/005

(57) ABSTRACT

Described herein is a method for packaging electrochemical reagents and enzymes into a low cost and compact ruggedized probe, suitable for use in harsh field conditions. A deep eutectic solvent replaces a portion of traditional aqueous buffer used to maintain enzyme activity: this elevates the boiling point, depresses the freezing point, and drastically lowers the vapor pressure and volatility of the buffer while still preserving enzyme activity and allowing diffusion of target vapors through the buffer. For example, phosphotriesterase or acetylcholinesterase enzymes can be used to detect organophosphates using electrochemistry.

### CROSS-REFERENCE TO RELATED APPLICATIONS

This application is related to U.S. Pat. Nos. 9,945,811 and 9,557,296.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; nrltechtran@us.navy.mil, referencing NC 211153.

### BACKGROUND

The use of enzymes can provide electrochemical assays with selectivity and specificity. However, most enzyme-based electrochemical probes struggle to survive conditions outside of the laboratory. For example, aqueous buffers can freeze or evaporate, inactivating the enzymes on which the assay depends. Furthermore, the lifetime of hydrated enzymes is typically short.

A need exists for new improved techniques for enzyme-based electrochemical assays.

### BRIEF SUMMARY

Described herein is a method for packaging electrochemical reagents and enzymes into a low cost and compact ruggedized probe, suitable for use in harsh field conditions. A deep eutectic solvent replaces a portion of traditional aqueous buffer used to maintain enzyme activity: this elevates the boiling point, depresses the freezing point, and drastically lowers the vapor pressure and volatility of the buffer while still preserving enzyme activity and allowing diffusion of target vapors through the buffer. A compact and low cost electrochemical assay probe includes a disposable screen printed electrode, enzyme, deep eutectic solvent, membrane separator, and an optional plastic separator that can be used to quickly rehydrate lyophilized or otherwise dry enzyme at the time of experiment. These probes are intended to be a disposable reagent package that can be used in any generic potentiostat, homebuilt or commercially available, that has been outfitted with a connector that can interface with a screen-printed electrode. When used with dry or lyophilized enzyme, they should have good shelf life and be amenable to room temperature storage.

In one embodiment, a biosensor includes a deep eutectic solvent; an electrode; an enzyme in contact with the deep eutectic solvent and the electrode; and a potentiostat operably connected to the electrode, wherein the enzyme is selected from the group consisting of phosphotriesterase and acetylcholinesterase.

In another embodiment, a method of sensing includes providing a biosensor according to the first embodiment, contacting the biosensor with a sample known or suspected to contain an organophosphate; and obtaining a measurement from the potentiostat, wherein the measurement correlates with the presence of organophosphate in the sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B provide schematics showing the different elements of the “turn on” PTE assay biosensor (FIG. 1A) and the “turn off” assay using acetylcholinesterase and the application of a redox mediator (FIG. 1B). FIG. 1C shows an exemplary electrode and nylon top membrane with a human fingertip (bottom) to provide a sense of the size of the device.

FIG. 2A is an illustration of an complete exemplary instrument including the circuit board, which runs both turn “on” and turn “off” assays simultaneously and two electrode probes for both assays mounted back to back. The entire package including both assay probes measures 2.5 cm×5 cm×1 cm with a weight of 10 grams. The potentiostat electronic components are located on the back side of the circuit board to protect them from accidental liquid spills; they are not visible in the drawing. Header pins allow for mounting directly onto a uncrewed aerial vehicle (UAV); they only supply raw power to the instrument and communicate data. FIG. 2B shows exemplary results of both assays running simultaneously when exposed to dimethyl methylphosphonate (DMMP) vapor, serving as a model organophosphate: Delta uA is depicted with a dashed line while Delta mV is shows as a solid line.

FIG. 3 depicts the optimization of the DES mixture and thin membrane for increased signal of DMMP.

FIGS. 4A and 4B depict two different DES formulations for the “turn off” assay: without (A) and with (B) acetylthiocholine chloride as part of the mixture. The deep eutectic solvent mixture 1 relating to FIG. 4A includes 1:2 choline chloride/gycerol, 100 mM potassium phosphate pH 7, and 100 mM acetylthiocholine iodide. The deep eutectic solvent mixture 2 relating to FIG. 4B includes 1:2 acetylthiocholine chloride/gycerol and 100 mM potassium phosphate pH 7.

FIGS. 5A and 5B show the temperature and altitude profile of a simulated flight and the results for the “turn on” assay comparing sensors after exposure to a simulated flight, respectivelya. The signal of the biosensor is in delta mV at 10 minutes time. The data was taking using a Kimwipe cellulose membrane with the DropSen 2^{nd} layer, 15 μL of the DES with 400 μg of OMV/PTE deposited on the RuO₂ electrode with or without Triton X-100.

FIG. 6A illustrates the construction of the probe ready for activation with a “pull-tab” design and storage while FIG. 6B depicts the activation of the probe.

FIG. 7A provides an exploded view of the probe design. FIG. 7B provides drawings of the activation of an actual probe. FIG. 7C shows data for DMMP detection after both immediate activation and for activation following storage for one month.

FIG. 8 shows exemplary DES components. By changing the chemical nature of the deep eutectic solvents, one can tune the physical and chemical properties to increase the sensitivity of the sensor by increasing diffusion and decreasing viscosity and increase partitioning into the sensor probe.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Overview

Electrochemical detection of organophosphates using water based buffers and immobilized enzymes on screen-printed electrodes have been reported for acetylcholinesterase (ACE) (see refs. 1-4) and phosphotriesterase (PTE) (see refs. 5-7), however the robustness of these biosensors for deployment and unattended operation in the field is very limited due to the fact that aqueous buffers can freeze or evaporate.

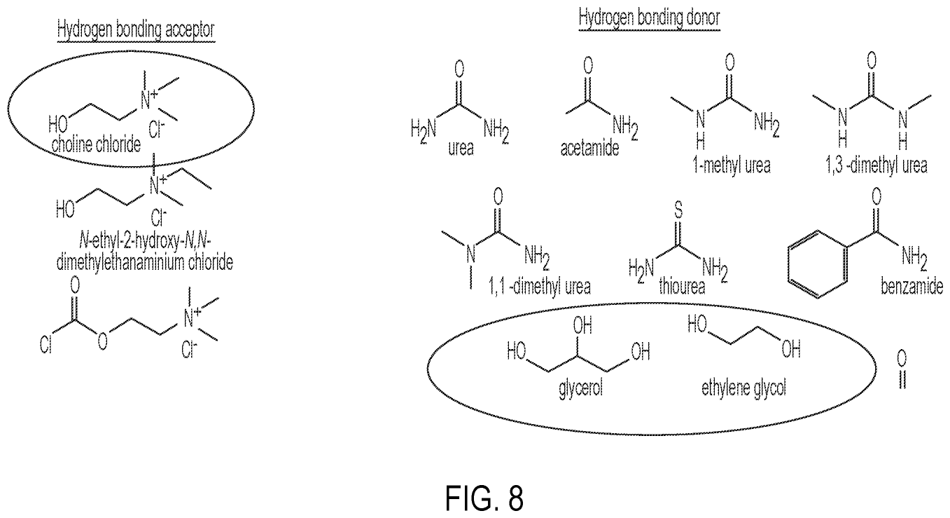

To mitigate this problem, one can add deep eutectic solvents (DES) that are mixtures of Lewis or Bronsted acids and bases that contain a variety of anionic and/or cationic species. This leads to a decrease in the freezing point of the mixture (see ref. 8). Accordingly, suitable DES form liquids at ambient temperatures from the formation of intermolecular hydrogen bonds. Deep eutectic solvents exhibit low vapor pressures, wide liquid temperature ranges, and are non-flammable. As described herein, DES can ruggedize enzymatic components of a biosensor, making it better suited to field use. FIG. 8 depicts a selection of some possible DES components.

Assays used in such biosensors can be grouped into two general categories: “turn on” assays where a readout is based on the product of the enzymatic reaction, and “turn off” assays where the readout is based on a decrease in enzymatic activity (such as by deactivation or inhibition of the enzyme).

As described below, two separate enzymes were packaged into ruggedized electrochemical probes to create a dual biosensor suitable for the field detection of organophosphate vapors in the field. The assay was tested and shown to work under a variety of environmental conditions using a homebuilt potentiostat. However, it is important to note that this technique is not restricted to these specific enzymes or particular use cases.

In each case, the electrochemical interface of the sensors are saturated with deep eutectic solvents which minimizes freezing or evaporation in extreme temperature and pressure range.

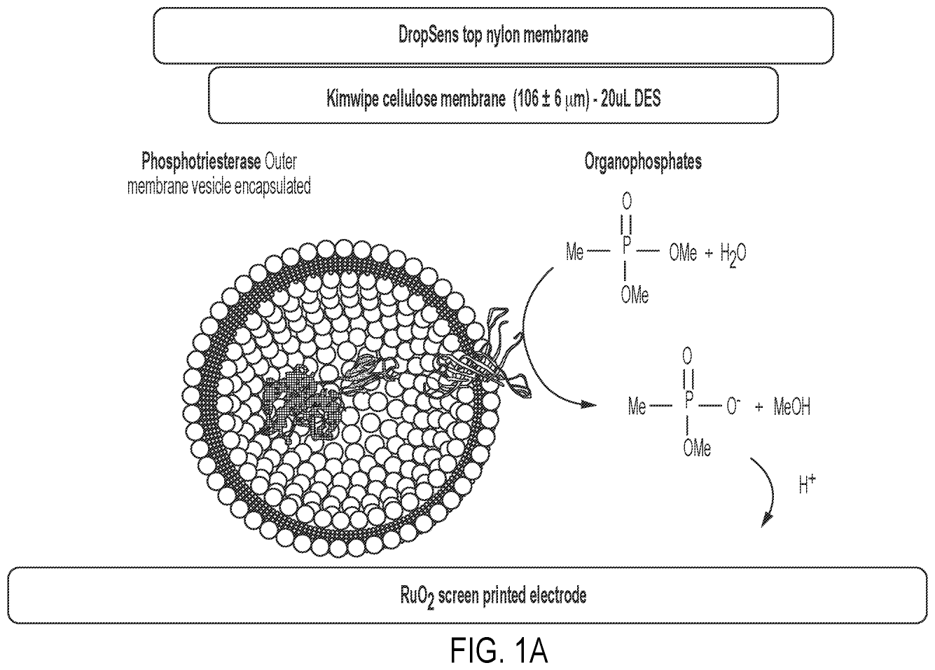

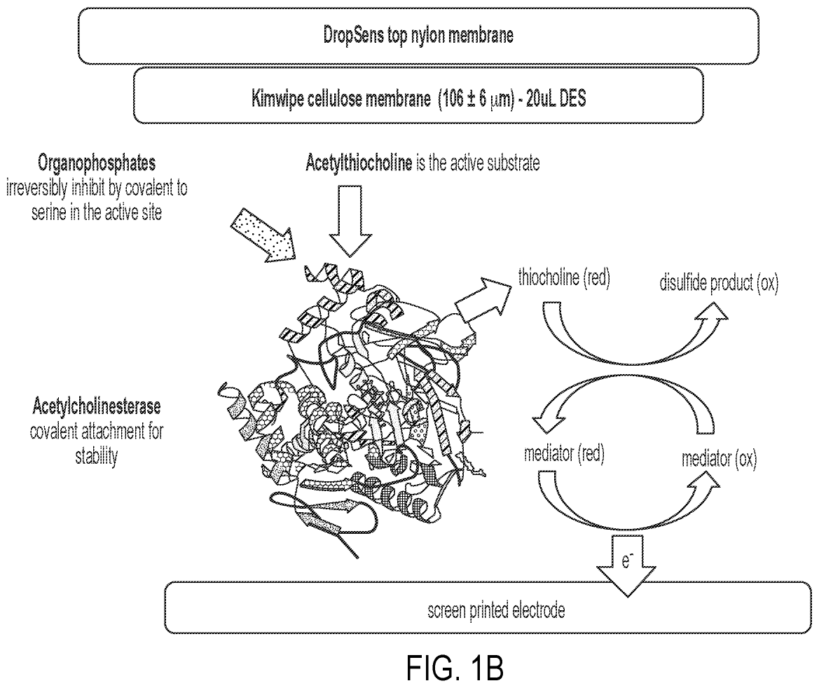

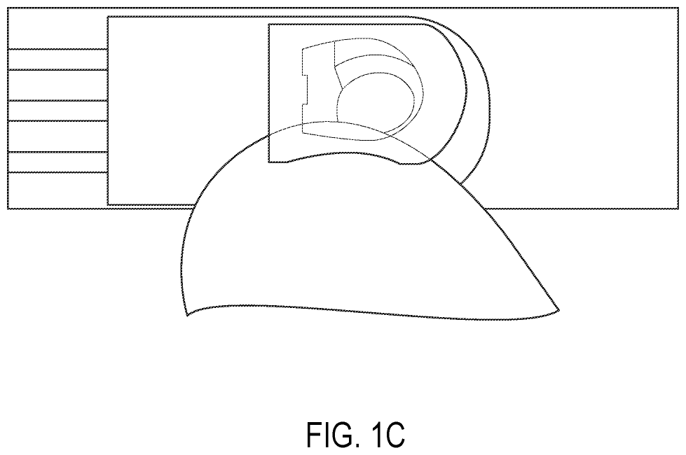

One exemplary turn on assay, for the detection of organophosphates (OP), is shown schematically in FIG. 1A. The enzyme phosphotriesterase (PTE) is encapsulated in outer membrane vesicles (OMV), termed OMV/PTE, as described in refs. 9 and 10, incorporated herein by reference for the purposes of describing techniques for making OMV/PTE. When exposed to OP, the vapor thereof partitions into the DES where the PTE hydrolyzes the organophosphate bond to generate protons which in turn is detected at a RuO₂ electrode as a change in voltage. This voltage serves as a readout to indicate the presence of OP.

Such a sensor can be constructed relatively simply, as was this example. The OMV/PTE was immobilized on a pH sensitive RuO₂ electrode and covered with a nylon membrane containing a small volume of a deep eutectic solution in a cellulose membrane as shown in FIG. 1C. In the presence of OP vapor, PTE hydrolyzes OP and generates protons. This activity can be quantified from the electrochemical detection of a pH change in mV between a reference electrode and a pH sensitive electrode.

An exemplary turn off assay, also for the detection of OP, is seen in FIG. 1B. The enzyme acetylcholinesterase is used in this case—the enzyme becomes inhibited by the covalent bonding of organophosphates to serine in the active site of the enzyme. In the presence of an active substrate actylthiocholine, the resulting change in electrochemical activity of the enzyme can be measured.

In electrochemical measurements, the overpotential is the potential difference (voltage) between a half-reaction's thermodynamically determined reduction potential and the potential at which the redox event is experimentally observed. In this case, one measures the activity of acetylcholinesterase by monitoring the oxidation of the enzyme product thiocholine. By adding a redox mediator, the overpotential needed at the electrode to observe the oxidation of the product can be lowered which decreases the background current and the noise level. In addition, a new DES is used which includes acetylthiocholine chloride as part of the DES mitigating the result of substrate depletion.

The exemplary sensor used a homebuilt potentiostat with a compact, light-weight design suitable for incorporation into a UAV. However, this technique could be used with practically any potentiostat, commercial or homebuilt, that has a connector capable of interfacing with a screen-printed electrode. The example instrument contains sufficient components to implement two distinct and complimentary measurements simultaneously: of course it may be possible to increase this number. Potentiometric measurements were performed with a dedicated circuit using a discrete instrument amplifier modified with additional electronic components. The goal is to detect a change in voltage between a proton sensitive electrode with the immobilized enzyme, phosphotriesterase (PTE) and a reference electrode as a function of time as the enzyme hydrolyzes the organophosphate, if present, to produce protons. The amperometric measurement (LMP 91000, Texas Instruments) measures a decrease in the current at the working electrode modified with acetylcholinesterase as a function of time as acetylcholinesterase becomes inactivated with organophosphate.

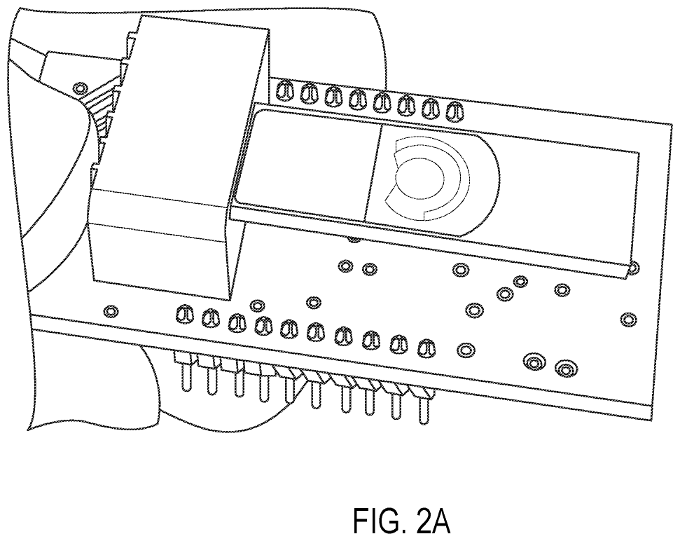

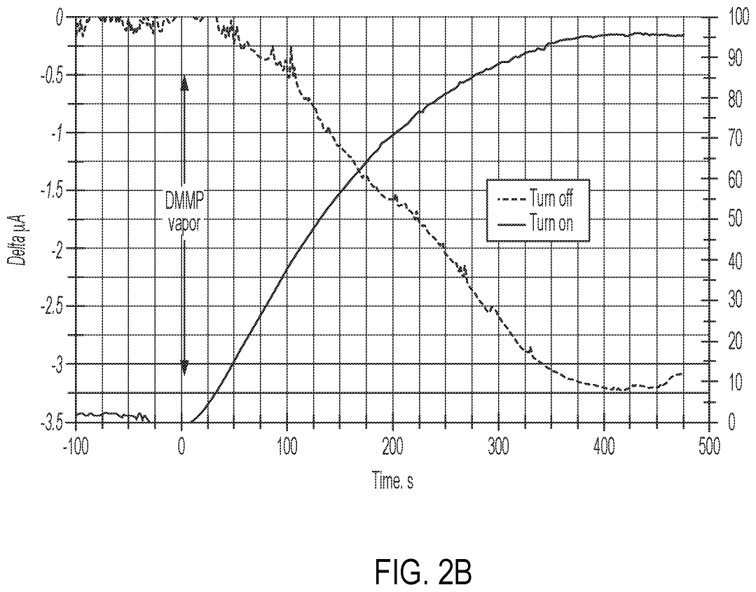

FIG. 2A shows the complete sensor unit. FIG. 2B is an example result of both assays running simultaneously detecting DMMP vapor. The size and weight of the sensor is small to facilitate mounting as a payload of a small UAV.

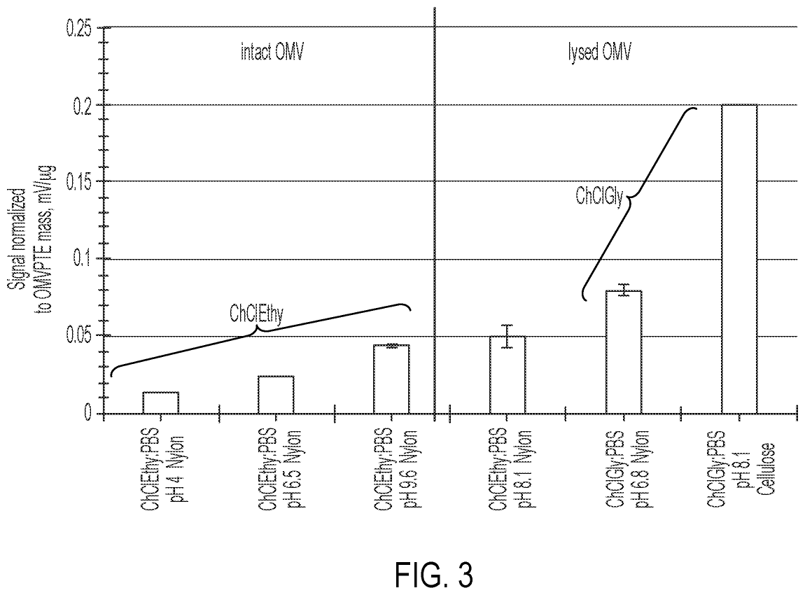

Novel formulations of deep eutectic solvents (DES) mixtures were explored. As shown in FIG. 3, by changing the DES from choline chloride with ethylene glycol (Ethaline) to choline chloride with glycerol (Glyceline), one sees a modest increase (60%) in signal from 15 mV to 25 mV when the biosensor is exposed to DMMP vapor. This may due to the different intrinsic pH values of each DES. Ethaline has a pH of 4, however glyceline has an intrinsic pH near 7. One sees an even greater increase in signal when changing the type of membrane on top of the RuO₂ which holds the DES. By changing the membrane from nylon to cellulose there is an increase 25 mV to 80 mV when the biosensor is exposed to DMMP vapor.

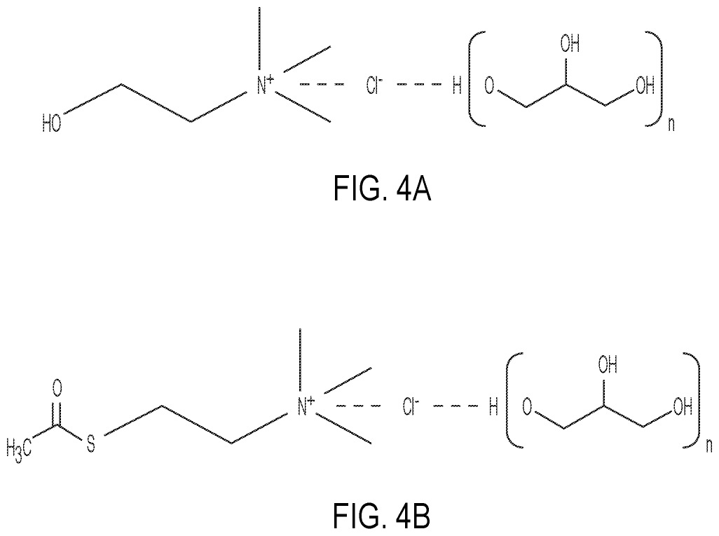

Acetylthiocholine chloride was explored as part of the DES. As shown in FIG. 1B, for the “turn off” assay, the activity of acetylcholinesterase is inactivated by the organophosphate and the activity of the enzyme i.e. the hydrolysis of acetylthiocholine chloride is monitored by the oxidation of the enzyme product thiocholine. A new DES was prepared using the enzymes substrate as a component of the DES mixture. The current DES was a 2:1 molar mixture of glycerol and choline chloride (FIG. 4A). For the new DES, the choline chloride was replaced with acetylthiocholine chloride, i.e. an enzyme substrate (FIG. 4B). This increases the available concentration of the substrate for long-term activation for field deployment.

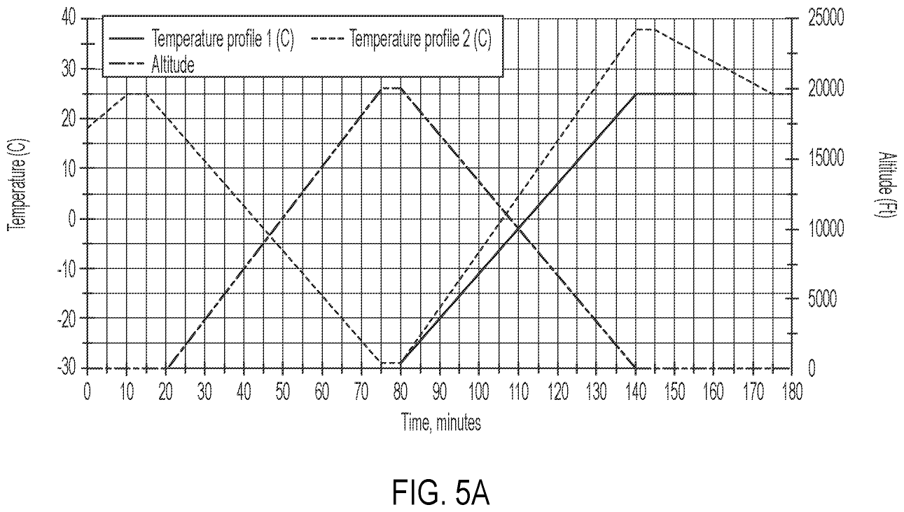

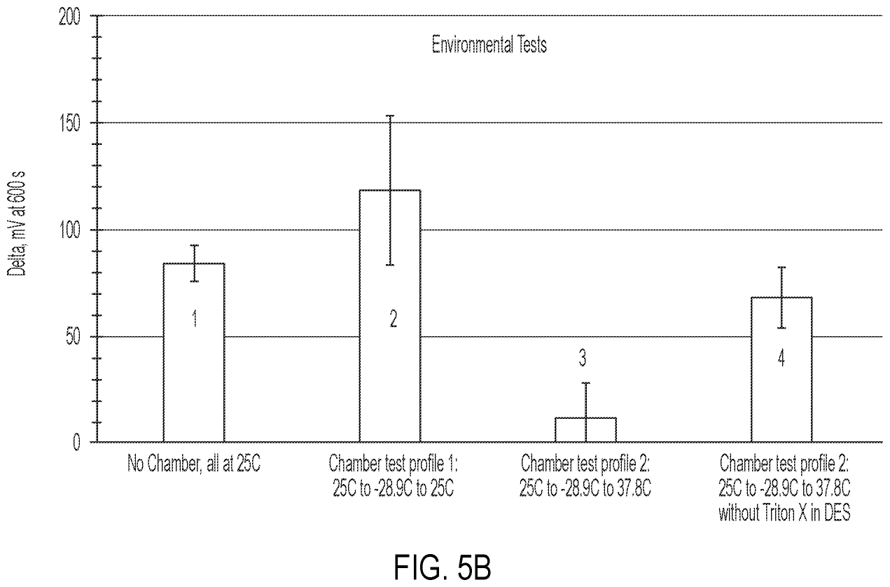

To evaluate the stability of the reagent package to harsh field conditions, testing was performed in an environmental chamber. A variety of altitudes along with harsh ground conditions were tested because this particular application was designed to be flown on a UAV. The temperature and pressure range simulated flight conditions starting at 20,000 ft with a temperature of −28.9 C (−20° F.), a pressure of 6 Psi and duration of 24 hours to ground desert conditions with the temperature at 48.9 C (120° F.), sea level ambient pressure. FIGS. 5A and 5B show two flight profiles and along with subsequent results of DMMP vapor tests.

Visible inspection of the reagent package after exposer to the flight profiles did not shown any crystallization, precipitation, nor significant separation of the layers, nor air bubble formation, thus suggesting that the good alignment between the layers and the tight configuration kept the layers was separating. Interestingly, the OMV/PTE enzyme improved its response time and amplitude (compared to controls) after it was exposed to the simulated flight in profile 1. It is suspected that there is some kind of constructive conditioning of the OMV/PTE and deep eutectic solvent when exposed to the low pressure and temperature. (FIG. 5B comparing bar 1 to bar 2). In addition, for profile 2, the signal at 600s was significantly improved when Triton X-100 was removed from the deposition protocol in the construction of the reagent package in FIG. 5B comparing bar 3 to bar 4. In summary, a combination of the low temperature and pressure and the absence of Triton X-100 restores the performance of the reagent package when exposed to the flight conditions in profile 2 with the hotter temperature.

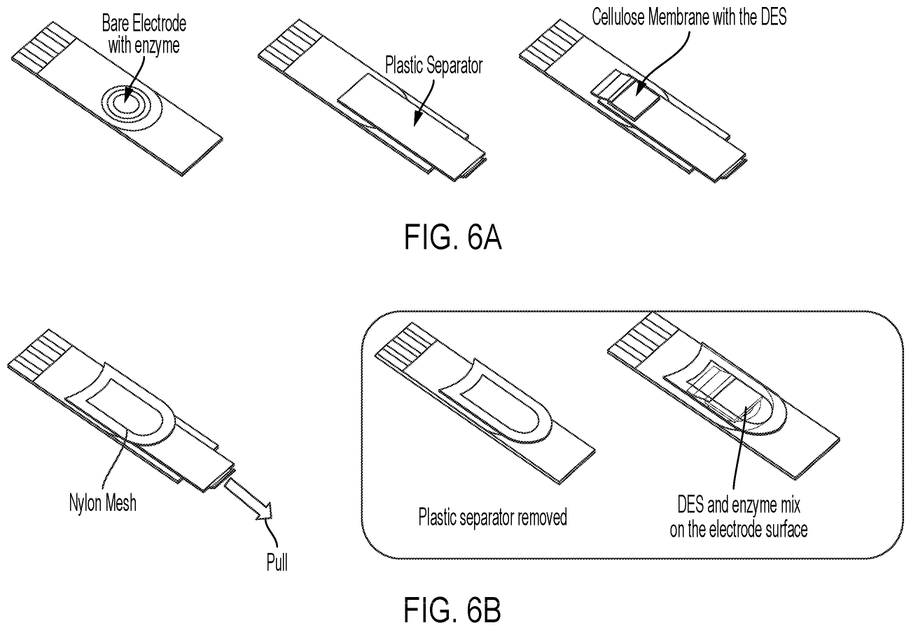

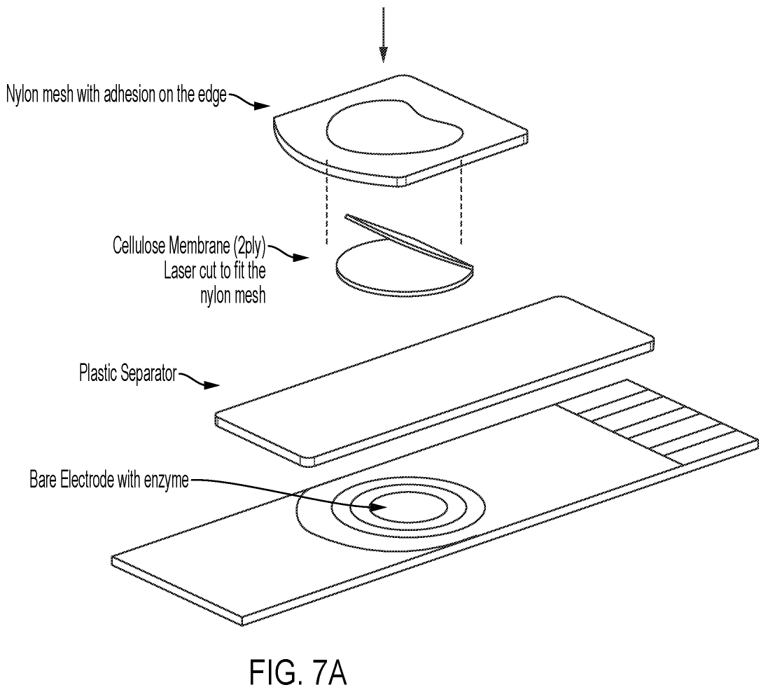

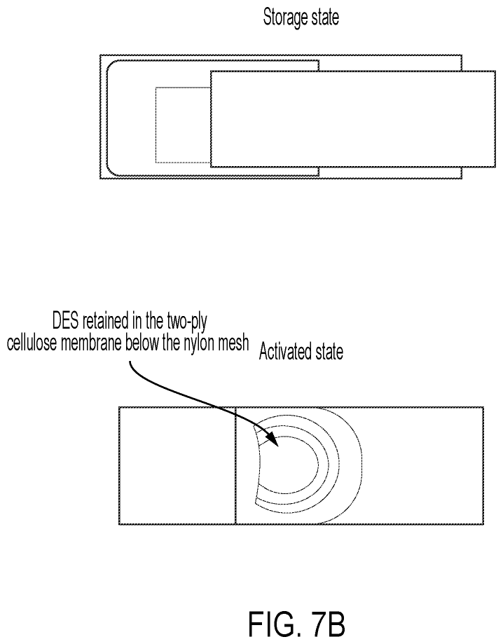

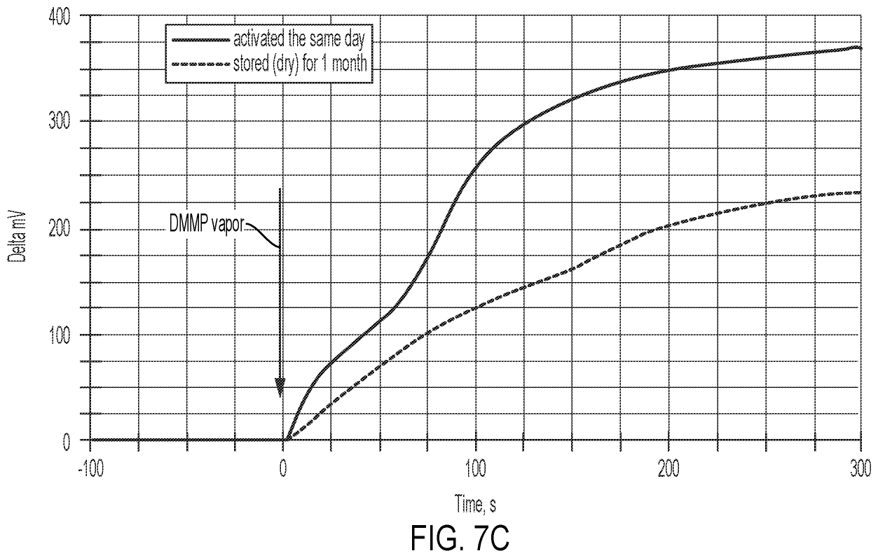

The sensor was designed for storage, easy activation and robustness for deployment in the field. The goal of the design of the reagent package is to facilitate easy of activation when deployed in the field and to further stabilize the liquid interface at the sensor surface. In FIGS. 6A and 6B, the parts and construction of the reagent package is as follows. In FIG. 6A, a plastic separator is placed top of the electrode modified with enzyme. Next the cellulose membrane which holds the deep eutectic solvent (DES) in placed on top of the plastic separator. In FIG. 6B, a nylon mesh with adhesion on the edges is used to seal all the layers on the electrode. For activation, the plastic separator is pulled to expose and mix the electrode surface/enzyme with the DES in the cellulose membrane.

Table 1 lists the parameters and materials for the construction of the reagent probe. Table 2 provides the analytical parameters for testing and the results. The second entree is the measurement made at the Vapor TestBed in the Chemistry Division. In this case, the DMMP vapor concentration was set to a trace level at 100 ppb with a constant flow of DMMP vapor impinging the sensor at 4 L per minute which gave a signal of 7.8 mV within 2 minutes with a large signal to noise ratio of 35.

TABLE 1
Construction of the sensor probe

| Materials | Turn “on” assay | Turn “off” assay |
|---|---|---|
| Electrode | RuO₂ | Carbon |
| Enzyme | 400 μg of lyophilized | 500 μg of lyophilized |
| | phosphotriesterase encapsulated | acetylcholinesterase re-suspended |
| | in outer membrane vesicles | in 25 μL of water with 1 mM |
| | (PTE/OMV) re-suspended in 10 | bis(sulfosuccinimidyl)suberate |
| | μL of water and then dropcasted | added to crosslink the enzyme and |
| | on to the electrode. | then dropcasted on to the |
| | | electrode. |
| DES | 1:1 of Choline Chloride/Glycerol | 1:1 of Choline |
| | (1:2) and PBS with 0.1% Triton X. | Chloride/Thiocholine |
| | Adjusted to pH 8. | Chloride/Glycerol (1:1:4) and |
| | | 0.25M Tris with 0.5 mM ferrocene |
| | | methanol at pH 8. |
| Layer for | cellulose membrane 2ply | cellulose membrane 1ply |
| the DES | (KimWipe) | (KimWipe) |
| Cover | Nylon mesh | Nylon mesh |

TABLE 2
Analytical parameters

| | Turn “on” | Turn “off” |
|---|---|---|
| Parameters | Delta mV | Delta μA |
| Vapor headspace of DMMP at 25 C. | 375^{a} | 3.25 |
| 100 ppb DMMP flowing at 4 L per minute | 7.8^{b} | — |

^{a}Measured in the vapor headspace of DMMP in a vapor box at 25 C. with the pull tab design at 300 s. Headspace vapor at 25 C. and 760 mmHg = 5562 mg/m³ or 1096 ppm.
^{b}Measured at the Vapor TestBed in the Chemistry Division. Parameters: DMMP = 100 ppb, flow rate = 4 L per minute at 25.2 C. absolute humidity = 13.1 g/m³.

### Further Embodiments

Encapsulation of phosphotriesterase in outer membrane vesicles should serve to increase enzyme stability for longtime storage and field operation. Although enzyme encapsulation is not required for this invention, it can be used to enhance the stability of the reagent probes.

Dual detection using two different enzymes provides confidence in the measurement by minimizing false positives and false negatives. Multiplexing to higher numbers of enzymes is straightforward.

Beyond the described examples, it is contemplated that practically any sensor based on an enzymatic reaction resulting in electrochemical activity could benefit from the described techniques. This may be particularly true of membrane-bound enzymes that could be encapsulated in vesicles.

### ADVANTAGES

Deep eutectic solvent mixtures minimize water evaporation and freezing. Novel formulations of deep eutectic solvents mixtures can improve sensor operation and sensitivity, as demonstrated in the specific use case for organophosphates detection. Thin membrane sensor probe for rapid vapor partitioning into the DES/enzyme layer for increased sensor response. A “pull-tab” probe design for easy activation and storage.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

ADDIN EN.REFLIST 1. Panraksa, Y.; Siangproh, W.; Khampieng, T.; Chailapakul, O.; Apilux, A., Paper-based amperometric sensor for determination of acetylcholinesterase using screen-printed graphene electrode. Talanta 2018, 178, 1017-1023.
2. Cinti, S.; Minotti, C.; Moscone, D.; Palleschi, G.; Arduini, F., Fully integrated ready-to-use paper-based electrochemical biosensor to detect nerve agents. Biosensors & bioelectronics 2017, 93, 46-51.
3. Stepankova, S.; Vorcakova, K., Cholinesterase-based biosensors. J Enzyme Inhib Med Chem 2016, 31 (sup3), 180-193.
4. Pohanka, M., Electrochemical Biosensors based on Acetylcholinesterase and Butyrylcholinesterase. A Review. International Journal of Electrochemical Science 2016, 7440-7452.
5. Songa, E. A.; Okonkwo, J. O., Recent approaches to improving selectivity and sensitivity of enzyme-based biosensors for organophosphorus pesticides: A review. Talanta 2016, 155, 289-304.
6. Kim, K.; Tsay, O. G.; Atwood, D. A.; Churchill, D. G., Destruction and detection of chemical warfare agents. Chem Rev 2011, 111 (9), 5345-403.
7. Mulchandani, P.; Mulchandani, A., Biosensor for direct determination of organophosphate nerve agents. Biosensors & bioelectronics 1999, (14), 77-85.
8. Pätzold, M.; Siebenhaller, S.; Kara, S.; Liese, A.; Syldatk, C.; Holtmann, D., Deep Eutectic Solvents as Efficient Solvents in Biocatalysis. Trends in Biotechnology 2019, 37 (9).
9. Alves, N. J.; Moore, M.; Johnson, B. J.; Dean, S. N.; Turner, K. B.; Medintz, I. L.; Walper, S. A., Environmental Decontamination of a Chemical Warfare Simulant Utilizing a Membrane Vesicle-Encapsulated Phosphotriesterase. ACS applied materials & interfaces 2018, 10 (18), 15712-15719.
10. Alves, N. J.; Turner, K. B.; Medintz, I. L.; Walper, S. A., Protecting enzymatic function through directed packaging into bacterial outer membrane vesicles. Sci Rep 2016, 6, 24866.

What is claimed is:

1. A biosensor comprising:
a deep eutectic solvent;
an electrode;
an enzyme in contact with the deep eutectic solvent and the electrode; and
a potentiostat operably connected to the electrode,
wherein the enzyme is selected from the group consisting of phosphotriesterase and acetylcholinesterase.

2. The biosensor of claim 1, wherein the electrode comprises RuO₂.

3. The biosensor of claim 1, wherein the deep eutectic solvent is selected from the group consisting of (1) glycerol and choline chloride and (2) glycerol and acetylthiocholine chloride.

4. The biosensor of claim 1, wherein the enzyme is the acetylcholinesterase and the deep eutectic solvent is (2) glycerol and acetylthiocholine chloride.

5. The biosensor of claim 1, comprising both phosphotriesterase and acetylcholinesterase each in contact with a different electrode.

6. An uncrewed aerial vehicle comprising
a biosensor comprising a deep eutectic solvent, an electrode, an enzyme in contact with the deep eutectic solvent and the electrode, and a potentiostat operably connected to the electrode, wherein the enzyme is selected from the group consisting of phosphotriesterase and acetylcholinesterase.

7. The uncrewed aerial of claim 6, wherein the electrode comprises RuO₂.

8. The uncrewed aerial of claim 6, wherein the deep eutectic solvent is selected from the group consisting of (1) glycerol and choline chloride and (2) glycerol and acetylthiocholine chloride.

9. The uncrewed aerial of claim 6, wherein the enzyme is the acetylcholinesterase and the deep eutectic solvent is (2) glycerol and acetylthiocholine chloride.

10. The uncrewed aerial of claim 6, comprising both phosphotriesterase and acetylcholinesterase each in contact with a different electrode.

11. A method of sensing comprising:
providing a biosensor comprising a deep eutectic solvent, an electrode, an enzyme in contact with the deep eutectic solvent and the electrode, and a potentiostat operably connected to the electrode, wherein the enzyme is selected from the group consisting of phosphotriesterase and acetylcholinesterase;
contacting the biosensor with a sample known or suspected to contain an organophosphate; and
obtaining a measurement from the potentiostat, wherein the measurement correlates with the presence of organophosphate in the sample.

12. The method of claim 11, wherein the electrode comprises RuO₂.

13. The method of claim 11, wherein the deep eutectic solvent is selected from the group consisting of (1) glycerol and choline chloride and (2) glycerol and acetylthiocholine chloride.

14. The method of claim 11, wherein the enzyme is the acetylcholinesterase and the deep eutectic solvent is (2) glycerol and acetylthiocholine chloride.

15. The method of claim 11, comprising both phosphotriesterase and acetylcholinesterase each in contact with a different electrode.
